(19) United States
(12) Patent Application Publication
(10) Pub. No.: US 2021/0308321 A1
(43) Pub. Date: Oct. 7, 2021

(54) BIODEGRADABLE POLYMER-CERAMIC BONE GRAFTS WITH OPEN SPIRAL STRUCTURES AND GRADIENT POROSITY AND METHODS FOR MAKING THEREOF

(71) Applicant: THE TRUSTEES OF THE STEVENS INSTITUTE OF TECHNOLOGY, Hoboken, NJ (US)

(72) Inventors: Alok Kumar, Baltimore, MD (US); Gan Zhou, Fair Lawn, NJ (US); Xiaojun Yu, Fishers, IN (US)

(73) Assignee: THE TRUSTEES OF THE STEVENS INSTITUTE OF TECHNOLOGY, Hoboken, NJ (US)

(21) Appl. No.: 17/221,730

(22) Filed: Apr. 2, 2021

Related U.S. Application Data
(60) Provisional application No. 63/004,461, filed on Apr. 2, 2020.

(51) Int. Cl. A61L 27/18; A61L 27/56; A61L 27/10; A61L 27/44; B33Y 80/00; A61F 2/28

(52) U.S. Cl. CPC A61L 27/18; A61L 27/56; A61L 27/10; A61F 2230/0091; B33Y 80/00; A61F 2/28; A61L 27/446

(57) ABSTRACT

A scaffold has a spiral configuration and gradient porosity designed to facilitate the healing of bone injuries. To make the scaffold, a sheet of polymeric material or the like is rolled into a spiral shape. In one embodiment, the resulting scaffold has an outer porous layer with high porosity, and a comparatively less porous inner layer in order to facilitate vascularization and promote recovery. The pores can be filled with a degradable polymer and/or growth factors, bioactive molecules, bactericidal drugs and/or other compositions to further promote recovery.

### CROSS-REFERENCE TO RELATED APPLICATION

This application claims priority to U.S. Provisional Patent Application Ser. No. 63/004,461 filed Apr. 2, 2020, the entire disclosure of which is incorporated herein by reference.

### STATEMENT OF GOVERNMENT INTEREST

This invention was made with government support under RO1 EB020640 awarded by the National Institute of Biomedical Imaging and Bioengineering of the National Institutes of Health. The government has certain rights in the invention.

### FIELD OF THE INVENTION

The present invention relates to bone scaffolds and, more specifically, to bone scaffolds that imitate the property of human bone and enhance recovery.

### BACKGROUND OF THE INVENTION

Each year more than 2.2 million surgeries are performed to treat bone defects worldwide, which costs roughly $2.5 billion. Typically, autogenous bone grafts are used to treat these defects. However, they often result in donor site morbidity. Allogeneic grafts are another treatment option. However, they are often very expensive and unsafe.

Due to their virtually unlimited supply, tissue-engineered scaffolds have shown to be a potential alternative to autogenous and allogenic bone grafts. Scaffolds having porous architectures with minimal surface area and high strength provide a requisite environment for nutrition supply and waste removal, as well as high bone ingrowth. However, it has been difficult to incorporate all of these parameters into a single scaffold due to technical challenges. For instance, porous scaffolds designed with present technological methods, such as 3D printing, are not very effective in promoting cell migration, both inside the pores and in the central region, due to the difficulty of access to the outer surface of the scaffold, owing to its relatively large depth. Therefore, such scaffolds have often failed to induce the bone ingrowth due to the limited nutrient supply caused by geometrical and structural constraints. This further leads to the migration of cells seeded inside the pores toward the surface, where the nutrient concentration tends to be higher than it is elsewhere. On the other hand, scaffolds having porous architectures are advantageous in that they have the ability to be loadable with various biodegradable biomaterials and growth factors, for the sustained and prolonged delivery of these bone-inducing factors to encourage the bone ingrowth and accelerate the healing of bone defects.

To address the foregoing considerations, spiral scaffolds have been developed in the past, using a solvent casting method. These spiral structures are useful due to their open structure, which allows easy cell migration, nutrient supply and metabolic waste removal. Furthermore, such spiral scaffolds can be used as a reservoir of growth factors, bioactive molecules, and bactericidal drugs, required for the complete healing of bone defect areas and osseointegration. However, spiral structures implemented in the past have suffered from poor mechanical properties and have offered less control over layer thickness and pore architecture within the layers. For these reasons, improved spiral bone scaffolds and associated manufacturing methods are desirable.

### SUMMARY OF THE INVENTION

To address the considerations laid out above, a 3D printing and solvent casting method for designing and manufacturing a biodegradable hybrid scaffold with minimal surface area and high strength has been developed. Spiral structures with minimal surface area (as compared to their solid counterparts) are fabricated by curling a 3D printed sheet of Poly(lactic acid) (PLA) with gradient pores.

As compared to conventional methods (casting and molding) of making spiral scaffolds where control over the thickness, number of layers, and layer architecture are difficult to attain, the present method of fabrication of spiral scaffolds allows for the precise control of layer architecture, layer thickness, and the number of layers. Also, this method allows for control over the interlayer spacing and scaffold diameter. These parameters are difficult to control using conventional methods (e.g., sintering, solution casting, and electrospinning) of spiral scaffold fabrication.

The inventive spiral-shaped construct with porous outer layers and a relatively solid core provides an ideal structure to withstand compressive loads, as well as to promote the vascularization through available surface pores. Furthermore, the PLGA5050-βTCP filling the pores helps in the delivery of beta tricalcium phosphate (i.e., (βTCP) at the defect area through the dissolution of PLGA, which helps the unique design achieve faster bone regeneration.

The developed spiral scaffold is designed with core layers having smaller pores and outer layers having bigger pores. In its intended application, the scaffold of the present invention will provide structural support to the bone defect area and promote bone formation under loading stress. The open spiral structure will enhance vascularization in bone graft. The inclusion of biodegradable materials and the incorporation of different growth factors and molecules can support various stages of bone development. In this context, three dimensional (3D) printed porous scaffolds have been found very promising due to their easy to create geometry with interconnected pores.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a better understanding of the present invention, reference is made to the following detailed description of various exemplary embodiments considered in conjunction with the accompanying drawings, in which like structures are referred to by the like reference numerals throughout the several views, and in which:

FIG. 1 is a CAD design image of a 3D sheet showing the presence of pores of different sizes in accordance with an embodiment of the present invention;

FIGS. 2A-2D are in the form of a flow diagram, showing the process of spiral scaffold fabrication using FDM and solvent casting methods, in accordance with an embodiment of the present invention, showing a 3D printed sheet of PLA with gradient porosity (see FIG. 2A), the application of 50PLGA-50beta TCP in ethyl acetate (see FIG. 2B), the curling of the sheet to make a spiral scaffold (see FIG. 2C) and the 3D spiral scaffold made of PLA/PLGA/beta TCP (see FIG. 2D);

FIG. 3 is a stress-strain diagram of 3DP PLA spiral scaffolds obtained from compression testing at room temperature;

FIG. 4 is a stress-strain diagram of 3DP PLA spiral-PLGA/beta TCP scaffolds obtained from compression testing at room temperature;

FIG. 5 represents the results of dissolution study, showing the efficacy of the present invention in the delivery of bioactive molecules;

FIGS. 6A-6D are scanning electron microscopy (SEM) images of 3DP PLA samples (6A and 6B) and 3DP PLA samples with PLGA in pores (6C and 6D) after 14 and 21 days of dissolution study, respectively;

FIG. 7A is a photograph of 3D printed sheets and a spiral scaffold of those sheets, as well as an inset image of an STL file, the text underneath the figure being present solely for contrast;

FIG. 7B is a representative SEM image of a 3D printed PLA sheet; and

FIGS. 7C-7D are SEM images depicting the sheet of FIG. 7B with its pores filled with PLGA5050-βTCP blend.

### DESCRIPTION OF EMBODIMENTS OF THE INVENTION

Embodiments are now discussed in more detail referring to the drawings that accompany the present application. In the accompanying drawings, like and/or corresponding elements are referred to by like reference numbers.

Various embodiments are disclosed herein; however, it is to be understood that the disclosed embodiments are merely illustrative of the disclosure that can be embodied in various forms. In addition, each of the examples given in connection with the various embodiments is intended to be illustrative, and not restrictive. Further, the figures are not necessarily to scale, and some features may be exaggerated to show details of particular components (and any size, material and similar details shown in the figures are intended to be illustrative and not restrictive). Therefore, specific structural and functional details disclosed herein are not to be interpreted as limiting, but merely as a representative basis for teaching one skilled in the art to variously employ the disclosed embodiments.

Subject matter will now be described more fully hereinafter with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein; exemplary embodiments are provided merely to be illustrative. Among other things, for example, subject matter may be embodied as methods, devices, components, or systems. The following detailed description is, therefore, not intended to be taken in a limiting sense.

Throughout the specification and/or claims, terms may have nuanced meanings suggested or implied in context beyond an explicitly stated meaning. Likewise, the phrase “in one embodiment” as used herein does not necessarily refer to the same embodiment and the phrases “in another embodiment” and “other embodiments” as used herein do not necessarily refer to a different embodiment. It is intended, for example, that covered or claimed subject matter include combinations of exemplary embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

New methods for fabrication of scaffolds having predefined pore architecture and distribution are presented. The uniquely designed hybrid spiral structure can be potentially used for the load-bearing applications to treat bone defects due to their: easy to fabricate method, flexibility to on-demand fabrication, ability to create patient-specific designs, high strength, relatively open structures for the bone ingrowth and vascularization, while offering improved biodegradability.

The developed method can also be translated to design neural conduits, scaffolds to treat osteochondral defects, and skin grafts for expedited healing.

In an exemplary embodiment of the present invention, a spiral-shaped hybrid structure of PLA/PLGA5050-βTCP can be designed and prepared. The designed hybrid structure is characterized by a 3D printed PLA scaffold with predefined pores (e.g., square-shaped), and these pores were filled with PLGA5050-βTCP (e.g., 1:1 ratio). The initial construct with no open pores provides mechanical strength. Importantly, the strength of the construct can be further tailored by the pore geometry. The dissolution of the fast degrading polymer PLGA5050 helps in the pore creation for further enhancing the tissue ingrowth and vascularization at a later stage. Moreover, βTCP helps in the mineralization by supplying the Ca²⁺ and PO₄³⁻.

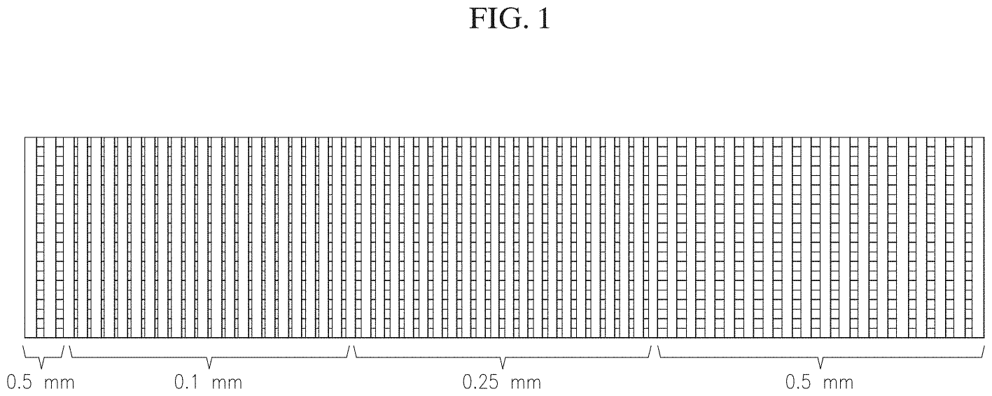

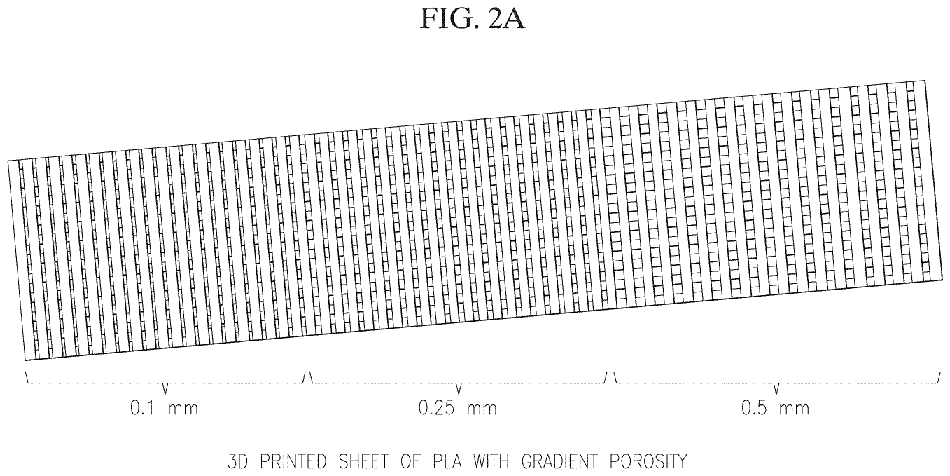

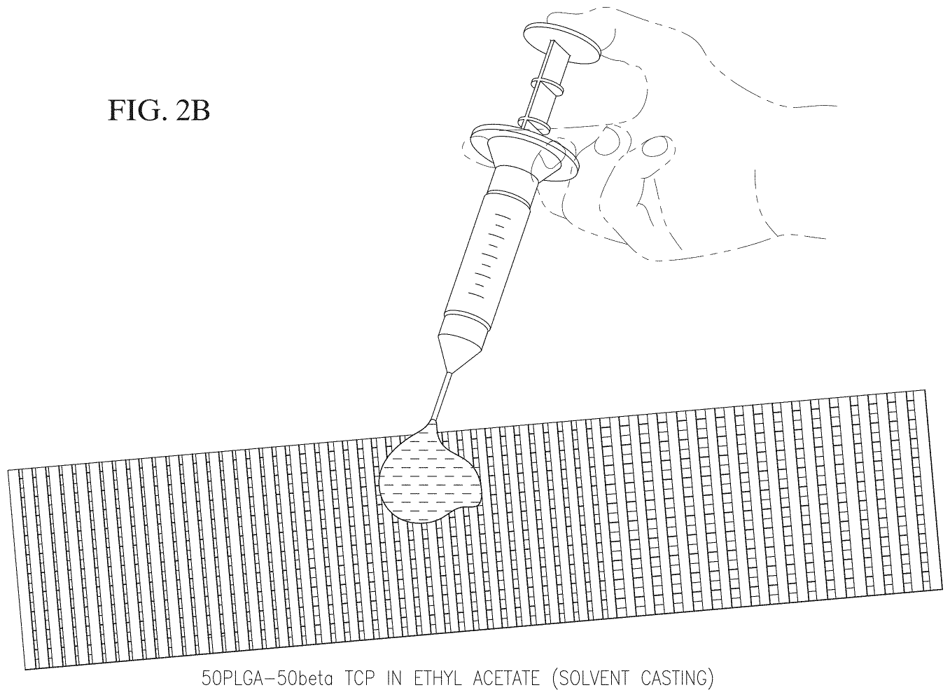

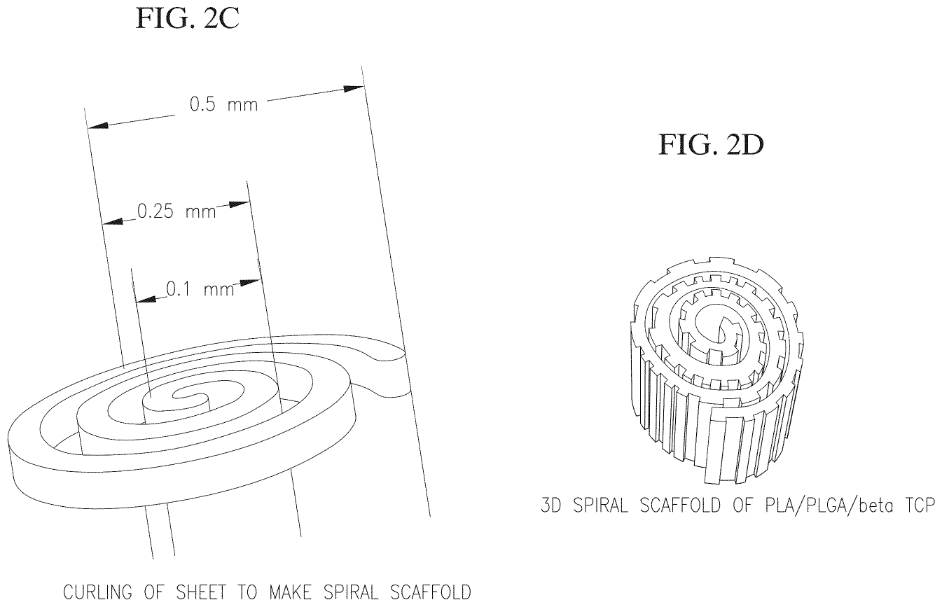

To fabricate the scaffold of the present invention, rectangular-shaped PLA sheets (e.g., 50 mmx 10 mmx 1 mm) with gradient porosity (see FIG. 1) were created using a fused deposition modeling (FDM) 3D printing method (FIG. 2). However, it should be understood that other sheet materials (e.g., polycaprolactone, collagen, etc.) can be used and that alternative 3D printing methods (e.g., robocasting, inkjet 3D powder printing, selective laser sintering, vat polymerization-based 3D printing, stereolithography, etc.) and non-3D printing techniques (e.g., polymer extrusion, compression molding, etc.) can be used to fabricate a scaffold in accordance with embodiments of the present invention. The sheet end with smaller pores was used in the core of the spiral scaffolds for improved compressive strength. The sheet end with bigger pores made the outer layers, ideal for the vascularization and bone tissue ingrowth. In an embodiment, 100% material infill density can be used. In an embodiment, the PLA sheet has 50% porosity. In other embodiments, to provide a gap between the layers in the curled (i.e., spiral) structure, an extra layer of PLA (e.g., of dimensions 10 mmx 0.1 mmx 0.1 mm (x)) may be added. The gap can be modified by changing the dimensions of the extra layer (e.g., by applying a multiplier “x”). This extra layer acts as a spacer between the layers of spiral structure.

The pores of the 3D printed PLA sheet can be filled with poly(lactic-co-glycolic acid) (PLGA) using a solvent casting method, in which the PLGA incorporates beta tricalcium phosphate (βTCP). Alternative methods for filling the pores of the present invention include dipping the scaffold in a biomaterial solution and/or via spraying the biomaterial on the structure. Moreover, the 3D printed porous structure can be filled with other biocompatible biomaterials or composite materials. Other variants of calcium-phosphate can also be incorporated. In this exemplary embodiment, the spiral structure and its size and shape provide structural support and act as a substrate for vascularization and osteogenesis.

An in vitro degradation study carried out at 37° C. revealed that the degradation of PLGA leads to release of βTCP particles. The results of this experiment suggest that the designed spiral scaffold with a fast degrading polymer filled in the pores of a PLA scaffold can be used for loading of growth proteins and molecules, and can further be used for delivery at the defect site in order to promote bone growth. Consequently, during the degradation of PLA, materials loaded within PLGA provide the cues for vascularization and new bone growth.

The ratio of PLGA and βTCP can be tailored to provide for optimal dissolution and bone growth. Furthermore, the mechanical strength and dissolution behavior of the scaffold can precisely and easily be tailored by modifying pore size and pore distribution. Moreover, layer thickness and the number of layers per unit diameter can be further used to adjust the mechanical, as well as dissolution, properties. In an embodiment, pores of these spiral layers can be filled with different drugs/growth factors loaded in PLGA to supplement the defect area during the healing process. In general, the designed pores act as a reservoir and therefore are only for the loading of materials required to promote bone growth. As such, not all pores are necessarily required to be filled with PLGA.

Subsequently the samples were dried and were curled into a spiral structure, followed by application of a heat treatment to improve their strength. A porous architecture was formed, characterized by the layers of spiral scaffold having different pore sizes.

The designed pores are a determining factor in mechanical properties and for inducing bone ingrowth. In general, smaller sized pores provide higher compressive strength, while pores with greater sizes are effective at enhancing vascularization. To exploit these properties, a sheet with gradient porosity was created. This sheet was curled in such a way that the smaller pores remain at the core of the spiral design, and relatively larger sized pores are present in the outer layers. Thus, this unique spiral design provides high axial strength to withstand the compression loading similar to cancellous bone, while at the same time providing the space for vascularization due to the open structure and surface pores.

Furthermore, the degradation of PLGA leads to the release and dissolution of βTCP particles, which supplements the defect area with calcium and phosphate ions, required for new bone formation.

As compared to conventional methods (e.g., casting and molding) for making spiral scaffolds, where the thickness, number of layers, and layer architecture are difficult to attain, the present method of fabrication of spiral scaffolds allows for the precise control of layer architecture, layer thickness, height, topography, interlayer spacing, scaffold diameter and the number of layers. These parameters are difficult to control using conventional methods of fabrication, including formation using sintering, solution casting, and electrospinning methods. Furthermore, the pores of the sheet can be filled with a variety of biodegradable biomaterials and growth factors for the sustained and prolonged delivery of these bone-inducing factors to encourage bone ingrowth and accelerate the healing of bone defects. In additional embodiments, layers of the spiral scaffold can be loaded with immunosuppressant drugs, antimicrobial substances, bone forming growth factors, angiogenesis promoting growth factors, etc., based on application requirements.

In an alternate embodiment, 3D printed structures without PLGA/βTCP can be formed with similar curling to make a spiral scaffold with relatively open space with respect to other embodiments.

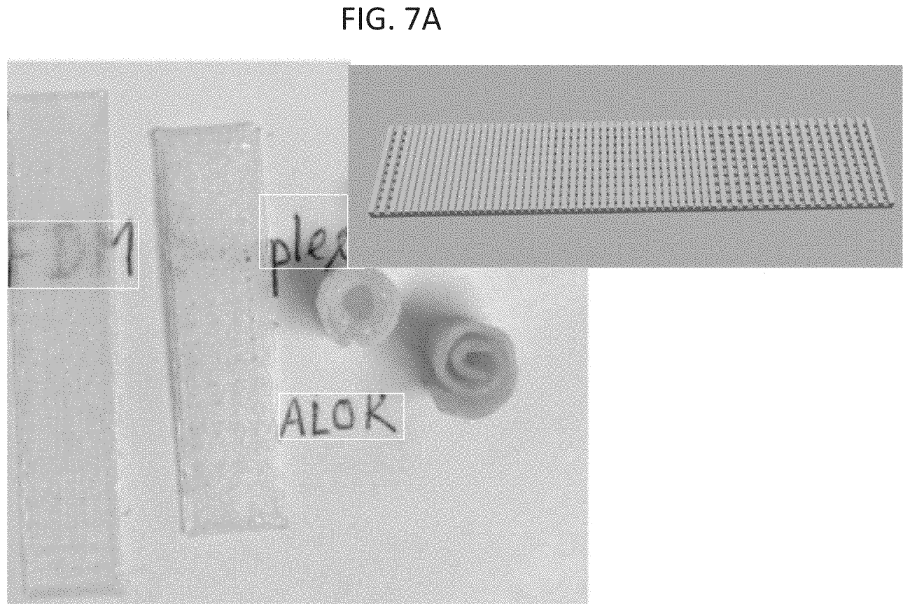

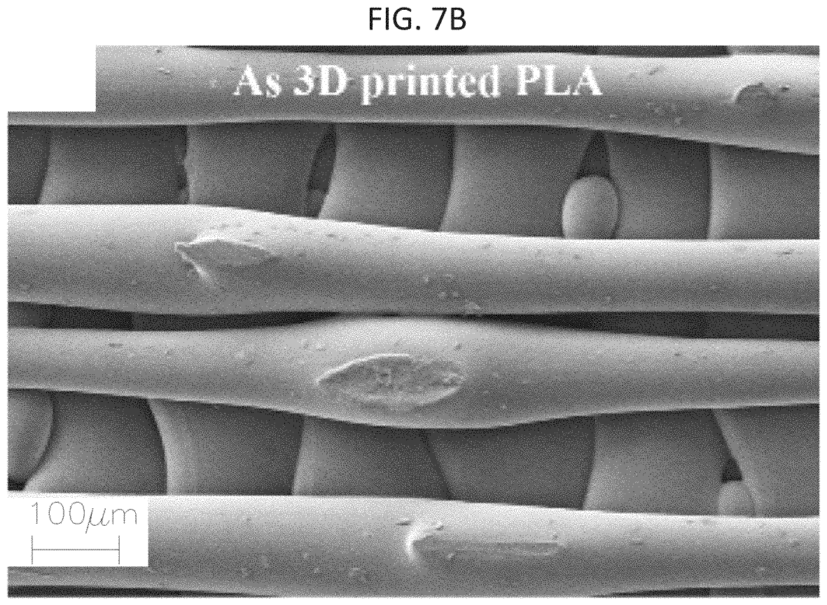

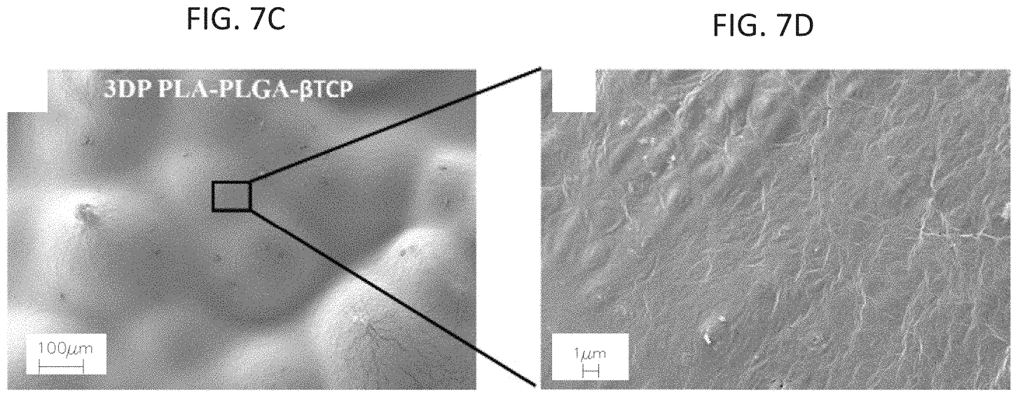

Example 1: Rectangular-shaped (50 mmx 10 mmx 1 mm) PLA scaffolds with ˜50% porosity were printed using an FDM printer (i.e., MakerBot Replicator) (FIG. 7A) with 100% material infill density. After the printing, the scaffold pores were filled with a blend of PLGA5050-βTCP by a solvent casting method. The dried composite structure of PLA/PLGA/βTCP was then curled into a spiral structure, followed by heat treatment to improve its strength.

An XRD and scanning electron microscope (SEM) were used for the phase and surface characterization of the as-sintered samples. Energy dispersive spectroscopy (EDS) was used for the elemental mapping and therefore, to detect the βTCP distribution in the scaffold matrix. For compressive strength analysis, cylindrical samples of 6 mm diameter and 12 mm height were tested using the universal testing machine. Furthermore, prepared samples were tested for degradability in 1×PBS at 37° C. for 2, 4, 6, 8 weeks.

The cylindrical-shaped spiral scaffolds were characterized by an open structure with a definite layer thickness (˜1 mm) and a height of 10 mm (FIG. 7A). The compositional analysis confirmed the presence of PLA, PLGA, and βTCP. Also, results showed the uniform distribution of βTCP in the PLGA matrix. The SEM revealed a highly porous structure, characterized by an average strut diameter of 250 μm (FIG. 7B). The PLGA5050-βTCP loaded 3D printed PLA scaffold showed a smooth surface with no open pores (FIGS. 7C, 7D). The mechanical strength of the spiral scaffolds was significantly improved. The dissolution of the PLGA/βTCP from the PLA matrix created the pores for tissue ingrowth and vascularization.

The uniquely designed hybrid spiral structure constructed via the combined use of 3D printing and solvent casting methods were defined by an open structure of PLA with designed pores, filled with PLGA5050-βTCP blend.

Example 2: A PLA filament of 1.77 mm diameter was used for the printing. 3D printing was completed at room temperature. Extrusion temperature and printing speed were 225° C. and 40 mm/s, respectively. Infill density was 100% with linear infill pattern.

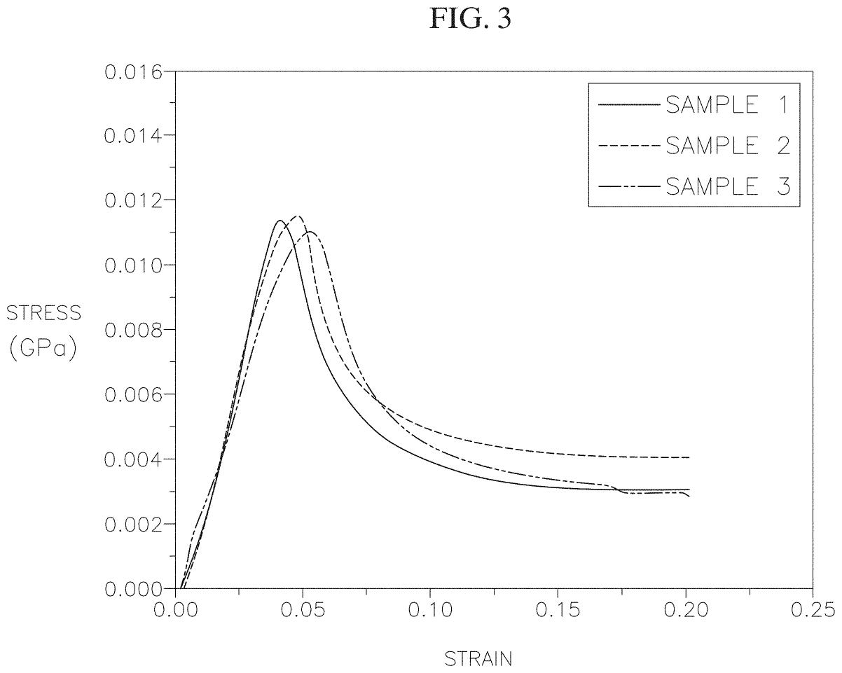

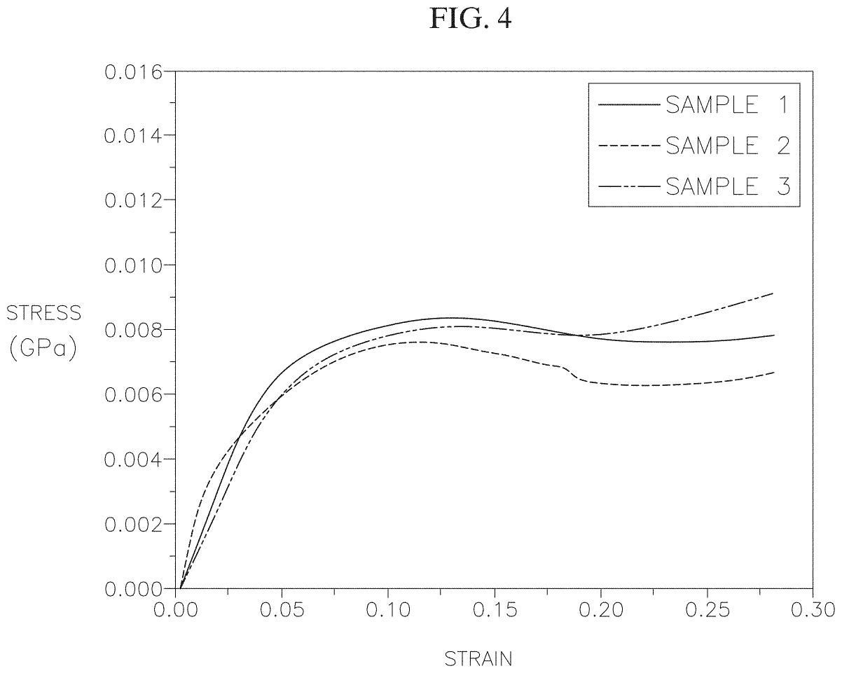

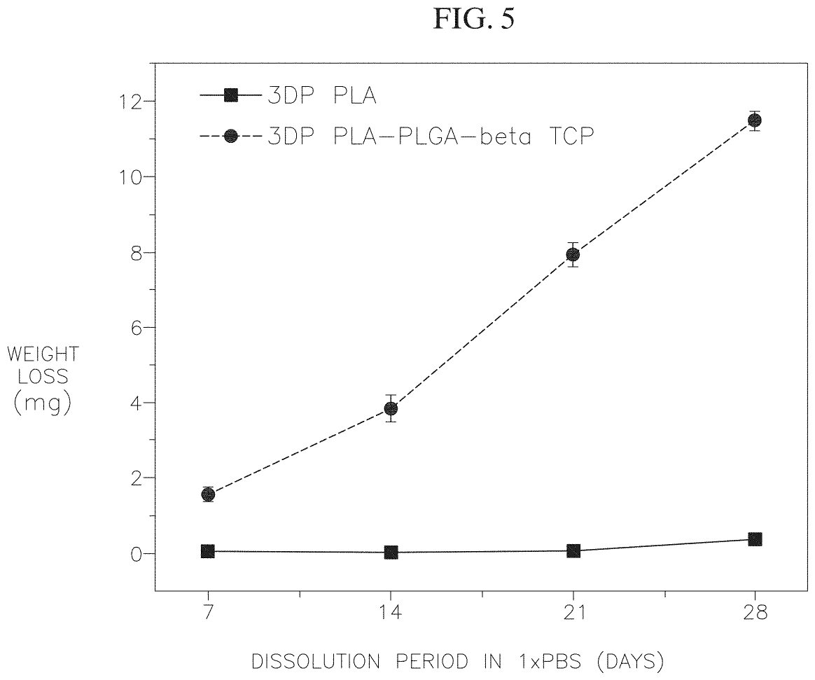

PLGA and beta TCP were mixed in a ratio of 50:50 at room temperature. Ethyl acetate was used as a solvent. This mixture was filled in the pores of 3D printed sheets, followed by overnight drying at room temperature. The dried sheet was heated in a hot air oven at 50° C. and curled to make a spiral construct. The pores were filled with 50PLGA-50beta TCP, followed by drying at room temperature. Mechanical testing of these samples confirmed compressive strength and elastic modulus in the ranges of 0.008±0.000 GPa and 0.193±0.033 GPa (FIG. 4), respectively. Similar testing was performed on an equivalent scaffold having just PLGA therein (FIG. 3). Furthermore, dissolution studies carried out in 1×PBS showed the removal of PLGA from the 3D printed PLA matrix and, therefore, the release of beta TCP in the vicinity of the scaffold. This availability of beta TCP in the defect area can promote bone growth. Importantly, no weight loss (degradation) in the PLA was noted during the dissolution period (28 days) (FIG. 5). This guarantees the stability of the scaffold structure after implantation. However, sustained dissolution of PLGA (with growth factors, drugs, and molecules) will provide the supplements for the bone growth while the pores remain open for facilitating vascularization.

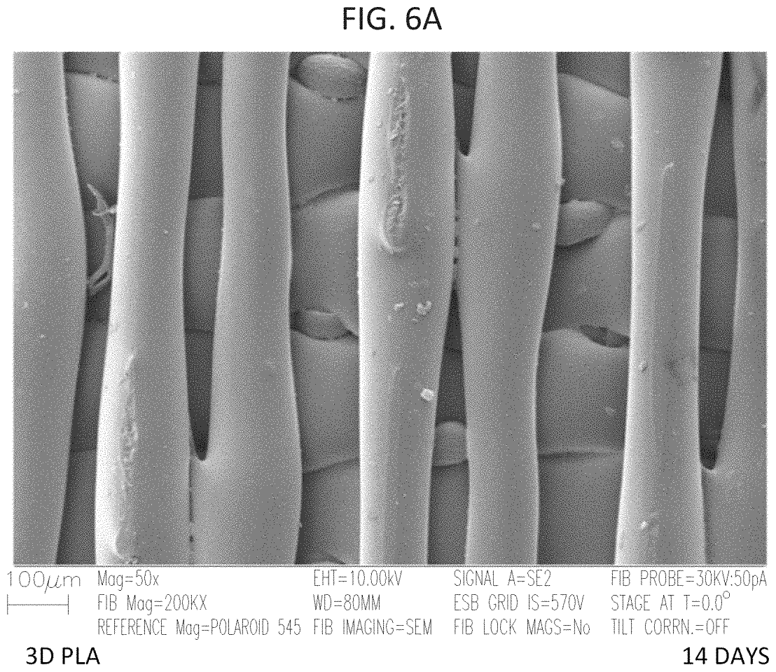

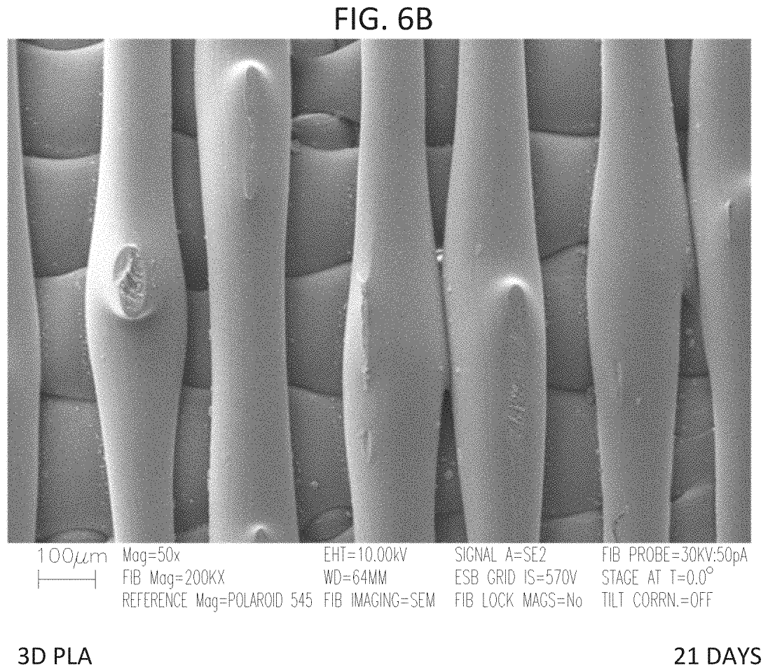

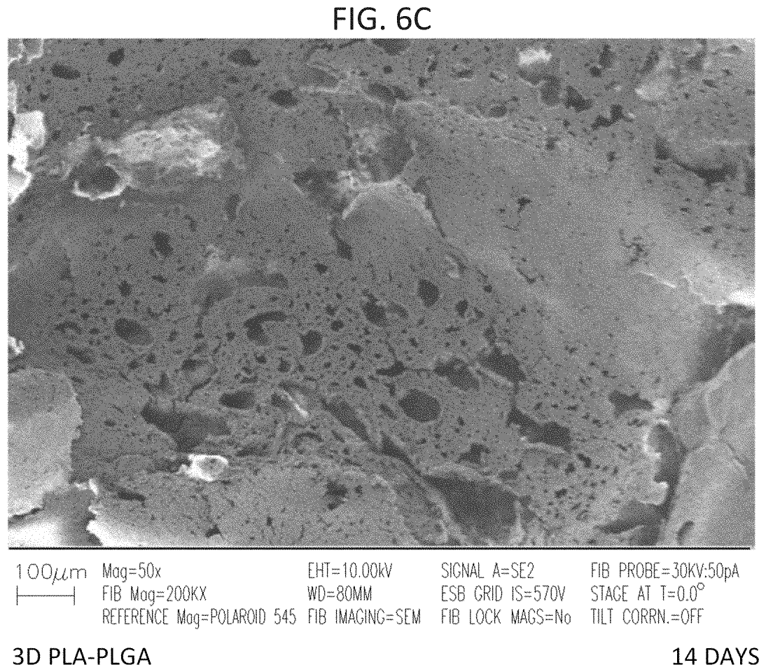

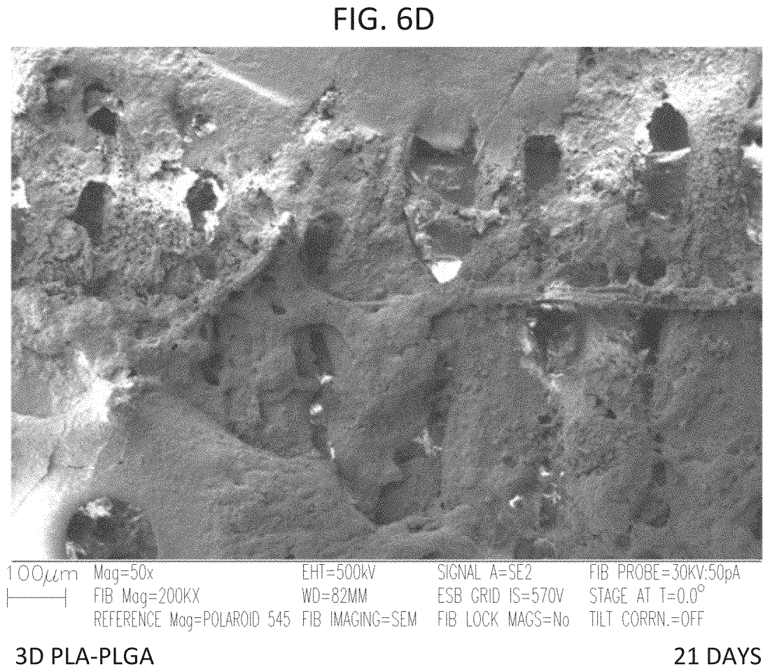

The 3D printed PLA samples, as well as PLA samples loaded with 50PLGA-50beta TCP, were analyzed by scanning electron microscope (SEM) for pore geometry and topography (FIGS. 6A-6D). Energy dispersive spectroscopy (EDS) was used for the elemental mapping and, therefore, to detect the βTCP distribution in the scaffold matrix. For compressive strength analysis, spiral samples of 12 mm height and 6 mm diameter were tested using the universal testing machine at 0.04 inch/min. Furthermore, prepared samples were tested for degradability in 1×PBS at 37° C. for 7, 14, 21, 28 days. The results showed degradation of PLGA with time; however, no such degradation was found in the PLA samples.

It will be understood that the embodiments described herein are merely exemplary and that a person skilled in the art may make many variations and modifications without departing from the spirit and scope of the invention. All such variations and modifications are intended to be included within the scope of the invention, as defined by the appended claims.

We claim:

1. A scaffold, comprising:
a body having a spiral configuration such that said body includes an inner layer and an outer layer;
a first plurality of pores provided in said inner layer such that said inner layer has a first porosity; and
a second plurality of pores provided in said outer layer such that said outer layer has a second porosity which is greater than said first porosity of said inner layer.

2. The scaffold of claim 1, wherein at least some of said pores of said first plurality of pores contain a polymer.

3. The scaffold of claim 1, wherein at least some of said pores of said second plurality of pores contain a polymer.

4. The scaffold of claim 1, wherein at least some of said pores of said first plurality of pores contain a polymer, and wherein at least some of said pores of said second plurality of pores contain said polymer.

5. The scaffold of claim 4, wherein said polymer fills said at least some of said pores of said first plurality of pores, and wherein said polymer fills at least some of said pores of said second plurality of pores.

6. The scaffold of claim 4, wherein said polymer fills all of said pores of said first plurality of pores, and wherein said polymer fills all of said pores of said second plurality of pores.

7. The scaffold of claim 4, wherein said body includes an intermediate layer between said inner layer and said outer layer.

8. The scaffold of claim 7, wherein said body has gradient porosity such that said intermediate layer includes a third plurality of pores providing said intermediate layer with a third porosity which is greater than said first porosity but less than said second porosity, at least some of said pores of said third plurality of pores containing said polymer.

9. The scaffold of claim 8, wherein said polymer fills all of said pores of said first plurality of pores, all of said pores of said second plurality of pores, and all of said pores of said third plurality of pores, whereby said scaffold has a composite construction.

10. The composite scaffold of claim 9, wherein said polymer is degradable.

11. The composite scaffold of claim 9, wherein said body has a polymer-ceramic composition.

12. The composite scaffold of claim 9, wherein said polymer comprises PLGA.

13. The composite scaffold of claim 12, wherein said polymer comprises PLGA5050-βTCP.

14. The scaffold of claim 1, wherein said pores of said first plurality of pores and said pores of said second plurality of pores are square in shape.

15. The scaffold of claim 1, wherein said body includes a rolled sheet of polylactic acid.

16. The scaffold of claim 1, wherein said pores of said first and second plurality of pores contain growth factors and molecules equipped to support specific stages of bone development and adapted to provide structural support to the bone defect area and promote bone formation under loading stress.

17. The scaffold of claim 1, wherein said body has an open spiral structure adapted to promote vascularization.

18. The scaffold of claim 1, wherein said pores of said first and second plurality of pores contain osseointegration factors.

19. The scaffold of claim 1, wherein said pores of said first and second plurality of pores contain bioactive molecules.

20. The scaffold of claim 1, wherein said pores of said first and second plurality of pores contain bactericidal drugs.

21. A method for fabricating a spiral scaffold, comprising the steps of
obtaining a sheet of rollable material;
providing a first segment of said sheet with a first plurality of pores such that said first segment has a first porosity;
providing a second segment of said sheet with a second plurality of pores such that said second segment has a second porosity which is greater than said first porosity of said first segment; and
rolling said sheet into a spiral configuration in which said first segment forms an inner layer of said scaffold and said second segment forms an outer layer of said scaffold.

22. The method of claim 21, wherein said sheet comprises polylactic acid.

23. The method of claim 21, further comprising the step of providing a third segment of said sheet with a third plurality of pores such that said third segment has a third porosity which is greater than said first porosity but less than said second porosity, said third segment being localized between said first segment and said second segment.

24. The method of claim 21, further comprising the steps of at least partially filling at least some of said pores of said first plurality of pores with a polymer and at least partially filling at least some of said pores of said second plurality of pores with said polymer.

25. The method of claim 24, filling step is conducted via a solvent casting method.

26. The method of claim 24, wherein said polymer comprises PLGA5050-βTCP.

27. The method of claim 24, wherein said polymer fills all of said pores of said first plurality of pores, and wherein said polymer fills all of said pores of said second plurality of pores.

28. The method of claim 24, wherein said polymer is degradable.

29. The method of claim 21, wherein said rollable material is obtained via a 3D printing process.

30. The method of claim 29, wherein said 3D printing process comprises a fused deposition modeling process.
